(19) United States
(12) Patent Application Publication
(10) Pub. No.: US 2022/0034761 A1
(43) Pub. Date: Feb. 3, 2022

(54) MICROBIAL SAMPLE COLLECTION, TRANSPORT AND PROCESSING APPARATUS AND METHOD

(71) Applicant: SHAZI S. IQBAL, SAN RAMON, CA (US)

(72) Inventor: SHAZI S. IQBAL, SAN RAMON, CA (US)

(21) Appl. No.: 17/233,366

(22) Filed: Apr. 16, 2021

Related U.S. Application Data
(60) Provisional application No. 63/058,425, filed on Jul. 29, 2020.

(51) Int. Cl. G01N 1/22; A61B 5/097; B01L 3/00; G01N 1/24

(52) U.S. Cl. CPC G01N 1/2205; A61B 5/097; B01L 3/502; G01N 2001/248; B01L 2200/142; B01L 2300/046; B01L 2300/0636; G01N 1/24

(57) ABSTRACT

A microbial sample collection apparatus includes a double-sided attachment member having opposed attachment surfaces such as adhesive surfaces. A sampling member adheres to one of the opposed surfaces of the double-sided attachment member. A remaining surface of the double-sided attachment member is attachable to a face covering, i.e. face mask, of a test subject. A sample collector adheres the sampling member to a selected location on a face mask interior surface via the double-sided adhesive attachment therebetween. In this manner, microbial samples are cumulatively collected from the exhalation breath path of the test subject wearing the face mask.

### BACKGROUND

The disclosures herein relate generally to the collection of microbial samples, and more particularly to the collection of microbial samples in a less intrusive and more efficient manner than provided by conventional microbial sample collection apparatus and methodology.

### BRIEF DESCRIPTION

In one embodiment, a microbial sample collection apparatus is disclosed that includes a double-sided attachment member having first and second attachment surfaces. The microbial sampling collection apparatus also includes a sampling member that is adhered to the first attachment surface of the double-sided attachment member. The microbial sample collection apparatus can include a facemask having an interior surface configured to face a test subject, wherein the sampling member adheres to a selected location on the face mask interior surface via the double-sided attachment member therebetween. In one embodiment, the double-sided attachment member is an adhesive dot that adheres the sampling member to the selected location on the face mask interior surface. In another embodiment, a first magnetic element adheres to the second attachment surface of the first double-sided attachment member, A second magnetic element is situated at an external surface of the facemask, wherein the magnetic force between the first and second magnetic elements that is exerted through the facemask is sufficiently strong to hold the sampling member with first magnetic element to the interior surface of the facemask at a predetermined location on the interior surface adjacent the second magnetic element on the exterior face mask surface. In one embodiment, the sampling member includes a negative control region that protects a portion of the sampling member from being exposed to microbes. In one embodiment, the sampling member includes a first filter layer situated atop a second filter layer, the first filter layer exhibiting a micron-rating sufficient to trap microbes larger than viruses such as bacteria while allowing viruses to pass therethrough, the second filter layer exhibiting a micron-rating sufficient to trap viruses or viruses in aerosols.

A microbial sample collection, transport and processing (MSCTP) container assembly is disclosed that includes an enclosure having an access region for adding and removing contents from the enclosure, the enclosure including at least one chamber therein for storing the contents. A double-sided attachment member is situated in the at least one chamber of the MSCTP container as contents thereof. A sampling member is also situated in the at least one chamber of the MSCTP container as contents thereof, the sampling member being configured to cumulatively collect microbial samples when installed on a selected surface via the double-sided attachment member.

In another embodiment, a method of processing samples is disclosed. The method includes placing, prior to microbial sample collection, a double-sided attachment member in a microbial sample collection, transport and processing (MSCTP) container, the MSCTP container including at least one chamber to receive the double-sided attachment member. The MSCTP container including an access portion that opens and closes to provide access to the at least one chamber. The method further includes, opening, in preparation for microbial sample collection, the access portion of the MSCTP container to remove the double-sided attachment member therefrom. The method further includes adhering a side of the double-sided attachment member to a sampling member. The method still further includes adhering another side of the double-sided attachment member to a surface within a breathing area of a subject such that the sampling member collects samples from the subject over a predetermined period of time, thus providing the sampling member with collected samples thereon. The method further includes moving the sampling member with samples thereon to the at least one chamber in the open MSCTP container. The method also includes closing the access portion of the open MSCTP container with the sampling member having microbial samples thereon inside the MSCTP container, thus providing a closed MSCPT container.

In yet another embodiment, a microbial sample collection assembly is disclosed that includes a double-sided adhesive member including first and second adhesive surfaces. The assembly includes a sampling member that is adhered to the first adhesive surface of the double-sided adhesive member. The assembly further includes a facemask including an interior surface configured to face a test subject, wherein the sampling member adheres to a selected location on the face mask interior surface via the double-sided adhesive member therebetween

### BRIEF DESCRIPTION OF THE DRAWINGS

The appended drawings illustrate only exemplary embodiments of the invention and therefore to not limit its scope because the inventive concepts lend themselves to other equally effective embodiments.

FIG. 1A is a perspective view of one embodiment of the disclosed MSCTP container.

FIG. 1B is a bottom view of the cap of one embodiment of the disclosed MSCTP container.

FIG. 1C is a top view of an alternative main body including two (2) chambers of one embodiment of the disclosed MSCTP container.

FIG. 1D is a top view of an alternative main body including four (4) chambers of one embodiment of the disclosed MSCTP container.

FIG. 1E is a top view of a double-sided attachment member associated with one embodiment of the disclosed MSCTP container.

FIG. 1F is a cross sectional view of the double-sided attachment member of FIG. 1E taken along line 1F-1F.

FIG. 1G is a top view of a sampling member associated with one embodiment of the disclosed MSCTP container.

FIG. 1H is a cross sectional view of a sampling member attached to a double-sided attachment member of one embodiment of the disclosed MSCTP container.

FIG. 1I is a cross sectional view of a sampling member that is attached via a double-sided attachment member to a surface in a test environment in accordance with one embodiment.

FIG. 2A is a side perspective view of another embodiment of the disclosed MSCTP container that exhibits a generally cylindrical geometry.

FIG. 2B is a bottom view of the top of one embodiment of the disclosed MSCTP container.

FIG. 2C is a top view of the main body of one embodiment of the disclosed MSCTP container.

FIG. 3A is a side perspective view of a generally parallelepiped-shaped embodiment of the disclosed MSCTP container shown in the closed state.

FIG. 3B is a top view of the disclosed MSCTP container of FIG. 3A that is shown in the open state.

FIG. 4A is a top view of a protective sleeve shown before the protective sleeve receives the sampling member.

FIG. 4B is a top view of the protective sleeve of FIG. 4A, but now depicted with the sampling member partially received by the protective sleeve.

FIG. 4C is a top view of the protective sleeve of FIG. 4A, but now depicted with the sampling member fully received by the protective sleeve.

FIG. 4D is a top view of an alternative protective sleeve wherein the sleeve's window is covered with mesh and is shown before the protective sleeve receives the sampling member.

FIG. 4E is a top view of the protective sleeve of FIG. 4D, but now depicted with the sampling member partially received by the protective sleeve.

FIG. 4F is a top view of the protective sleeve of FIG. 4D, but now depicted with the sampling member fully received by the protective sleeve.

FIG. 4G is a top view of a protective sleeve that is made from a mesh material throughout.

FIG. 4H is a top view of the protective sleeve of FIG. 4G wherein the sampling member is fully inserted in the protective sleeve.

FIG. 4I is a cross sectional view of the protective sleeve of FIG. 4F taken along section line 4I-41.

FIG. 4J is an end view of a multi-layered sampling member.

FIG. 5A is a perspective view of an alternative microbial sample collection transport and processing container (MSCTP).

FIG. 5B is a top view of a representative double-sided attachment member.

FIG. 5C is a top view of a representative sampling member.

FIG. 5D is a perspective view the MSCTP container with double-sided sampling member and sampling member therein while in the open state.

FIG. 5E is a perspective view the MSCTP container with double-sided sampling member and sampling member therein while in the sealed closed state.

FIG. 5F is a top view of a representative circular double-sided attachment member and a representative circular sampling member.

FIG. 5G is a perspective view of a face mask with a sampling member installed on the interior surface thereof.

FIG. 5H is a perspective view of a face mask with a sampling member installed on the exterior surface thereof.

FIG. 5I is a perspective view of a face mask with a sampling member installed at the center of the interior surface thereof.

FIG. 5J is an end view of a sampling member attached via a double-sided attachment member to a face mask in one embodiment.

FIG. 5K is an end view of a double-sided adhesive dot that acts as the double-sided attachment member in one embodiment.

FIG. 5L is an end view of a sampling member attached to a face mask via the double-sided adhesive dot of FIG. 4K.

FIG. 5M is an end view of a multilayer filter sampling member attached to a face mask via a double-sided adhesive member.

FIG. 5N is an end view of a multilayer filter sampling member attached to a face mask via the double-sided adhesive dot of FIG. 4K.

FIG. 5O is an end view of a multilayer filter sampling member attached to the internal surface of a face mask via magnetic elements.

FIG. 5P is an end view of a multilayer filter sampling member attached to the external surface of a face mask via magnetic elements.

FIG. 5Q is an end view of another embodiment wherein a multilayer filter sampling member is attached to the internal surface of a face mask via magnetic elements and another multilayer filter sampling member is attached to the external surface of the same face mask via magnetic elements.

FIG. 5R is an end view of another embodiment wherein a multilayer filter sampling member is attached to a face mask via magnetic elements.

FIG. 6 is a flowchart that depicts one embodiment of process flow in the disclosed sample collection, transport and processing methodology.

### DETAILED DESCRIPTION

In one embodiment, a microbial sample collection, transport and processing (MSCTP) container is disclosed that solves many of the problems associated with conventional microbial sample collection from individuals. The very significant problems encountered with such microbial sample collection are discussed below. Microbial sample collection may also be called microbial specimen collection in this description.

Microbial samples can be collected by methods such as taking a swab of the subject's nose or taking a swab of a subject's throat. These methods are of course very intrusive to the subject and require 1) sending the swab to the sample collector, 2) the sample collector inserting the swab into the subject's body to collect the sample, 3) the sample collector packaging the collected sample, and 4) the sample collector sending the packaged sample to a test facility where the sample is tested to determine if the sample includes a particular microbe or particular microbes. Unfortunately, with respect to asymptomatic carriers, the quantities of microbes available at the time of swab collection may be so low that swabbing the carrier's throat or mouth results in the test yielding a falsely negative result compared to a cumulative sample that is being collected over a number of hours by continuously monitoring shedding of microbes through aerosols released during breathing, talking or coughing, as disclosed in the methodology herein.

In the typical workplace environment during a pandemic or other widespread disease outbreak, employees are concerned with potentially being exposed to a microbe while working in the workplace environment. If an employer attempts to provide employees with swab testing at the workplace, employees who are asymptomatic may decline to be tested because they believe that they are not sick or for other reasons. While employees in the workplace environment may be required to wear a mask by a governmental entity or the employer, many employees are nonetheless still concerned with being exposed to microbes, especially from asymptomatic co-workers who do not even know that they are disease carriers or the environment in general. Outside the workplace, individuals are still concerned with potentially being exposed to microbes in environments such as hotels, airplanes, auditoriums and other places that individuals frequent.

The disclosed microbial sample collection, transport and processing (MSCTP) container provides a very convenient way to: 1) provide a sample collector with materials needed to acquire a microbial sample from a subject in a less intrusive and more reliable manner than other methods, 2) to store the acquired microbial sample while assuring sample integrity in a contamination-free manner, and 3) to transport the microbial sample to a testing facility. In one embodiment, the MSCTP container also provides a convenient sealed chamber in which initial processing of the microbial sample may commence as discussed in more detail below.

### COMPONENT LIST

The following component list is provided as a convenience to the reader. The disclosed technology is not limited to only these components that are recited below for purposes of example.
100 MSCTP container
105 Main body
110 Screw-on cap
110A Identification region
115 Threads
120 Threads
121 Chamber
125, 125′, 125″, 125′″ Double-sided attachment members
125-P Double-sided attachment member
130-P Sampling member
130A-P, 130B-P Sampling member
122, 123 Chambers
126, 127, 128, 129 Chambers
130, 130′ Sampling member
130A upper surface of sampling member
130B Lower surface of sampling member
130C Perimeter of sampling member
131 Coarse filter
132 Fine filter
135 Partition wall
150 Flexible substrate
155, 157 Adhesive layers
158, 159 Removable backing layers (release liners)
160 Negative control region
165 Surface in the test environment
200 MSCTP container
205 Main body
205A Outer main body wall
207 Cap
210 Snap-on cap
210A Interior snap-on cap wall
215 Chamber
300 MSCTP container
305 Top portion
310 Bottom portion
315 Hinge
320 Latch
320A Upper latch
320B Lower latch
325 Chamber
330 Upper gasket
335 Lower gasket
405 Protective sleeve
405A Front Panel
405B Back Panel
405C, 405D, 405E Closed side edges
405F Open side edge
410 Window
415 Mesh
420 Arrow
425 Adhesive member
430 Adhesive member
500 MSCTP container
502 Front panel
502A Seal
504 Back panel
504A Seal
505 Opening
550 Face mask
550A Interior of face mask
550B Exterior of face mask
570, 580 Magnetic elements
550A-P Interior of face mask
550B-P Exterior of face mask
570-P, 580-P Magnetic elements
590 Adhesive dot

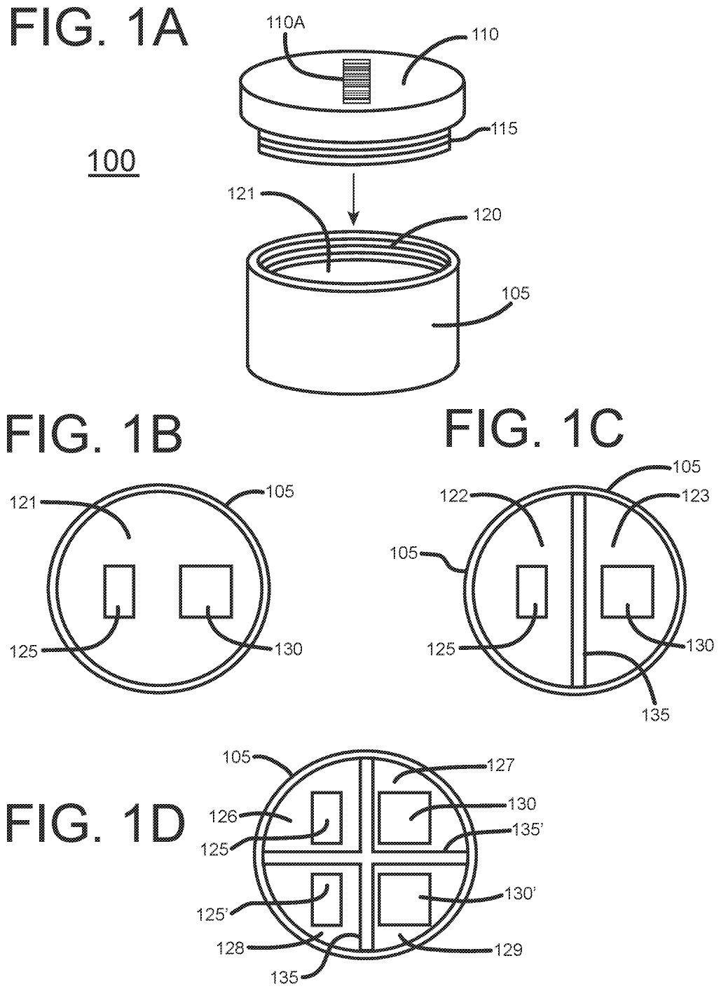

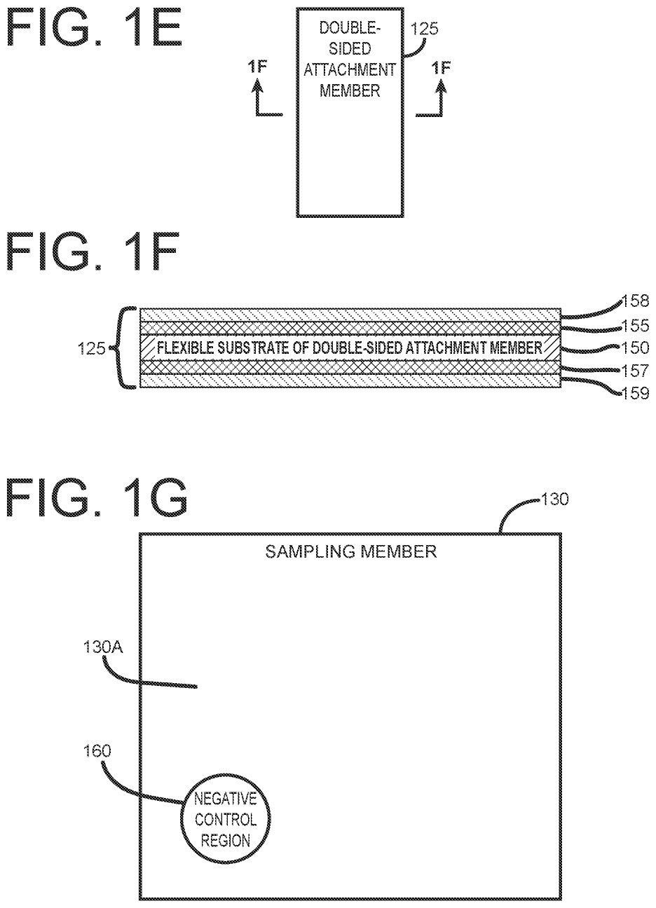

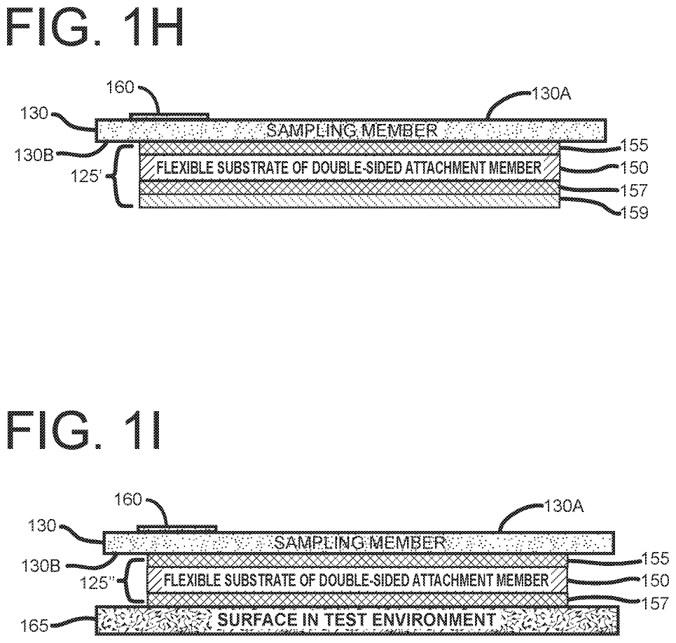

FIG. 1A is a perspective view of one embodiment of the disclosed MSCTP container 100 that includes a main body 105 and a screw-on cap 110. In this particular embodiment, MSCTP container 100 exhibits a generally cylindrical geometry, while other geometries may be employed as well. Screw-on cap 110 includes threads 115 that mate with and screw into threads 120 in main body 105 to sealably close MSCTP container 100. As seen in the top view of the open main body 105 of FIG. 1B, main body 105 includes at least one chamber 121 that receives microbial sample gathering related articles such as a double-sided attachment member 125. By way of example and not limitation, when cap 110 is fully screwed on main body 105, the vertical height of MSCTP 100 is approximately 15 mm and the diameter of main body 105 is approximately 45 mm. One embodiment of double-sided attachment member 125 is seen in FIGS. 1E-1G which is discussed in more detail below.

Cap 110 and/or main body 105 includes an identification region 110A that includes indicia of the identity of the subject of the test when an individual is being tested for the presence of microbes. For example, identification region 110A may include a bar code, a QR code, or a printed or handwritten name that may identify the test subject, date, time sample collected, sample collector identification, location where collected and type of sample. Alternatively, when sampling member 130 is not being used to test an individual for microbes but is instead being used to test for the presence of microbes at a particular location in an environment of interest, then identification region 110A identifies that location.

The term “sample collector” generally refers to the person who installs the sampling member 130 on double-sided attachment member 125, and who installs this sample gathering assembly on a selected surface for which cumulative microbial sample collection over time is desired. For example, the sample collector may install the assembly on the interior surface of a face mask for cumulatively collecting microbial samples directly from the subject's breath path. The sample collector may install the sample gathering assembly on other surfaces as well, as described below. In one scenario the sample collector may refer to the subjects themselves if they perform the above-described installation instead of someone else. However, if a person other than the subject applies the sampling member to a surface to collect a sample, then the sample collector refers to the person installing the sampling member, not the subject.

As seen in FIG. 1B, the chamber 121 that holds double-sided attachment member 125 also holds a sampling member 130. Sampling member 130 is any structure capable of collecting microbial samples, such as microbes or other microorganism samples. For example, sampling member 130 may be a paper material, filter material, membrane material or other surface on which microbial samples can gather for collection purposes. Sampling member 130 may be fabricated of paper, gelatin, synthetic plastic fibers such as polypropylene, nitrocellulose, nanocellulose, and polymer fibers such as Nylon and Rayon polyester. As seen in FIG. 1B, the chamber 121 that holds double-sided attachment member 125 also holds sampling member 130. In FIG. 1B, sampling member 130 need not be pre-attached to double-sided attachment member 125. However, for each of use, sampling member 130 may optionally be pre-attached to one side of double-sided attachment member 125 such that sampling member 130 is ready for immediate use upon the arrival of MSCTP container 100 at the location of the sample collector.

As seen in the embodiment of FIG. 1C, a partition wall 135 extends from interior side to interior side of main body 105 to divide the interior into chambers 122 and 123. Double-sided attachment member 125 and sampling member 130 are situated in chambers 122 and 123, respectively,

In yet another embodiment seen in FIG. 1D, a partition wall 135 extends from interior side to interior side of main body 105, and a partition wall 135′ extends from interior wall to interior wall of main body 105 as depicted. Partition walls 135 and 135′ intersect at the center of main body 105 to divide main body 105 into chambers 126, 127, 128 and 129. Double-sided attachment members 125 and 125′ are situated in chambers 126 and 128, respectively. Sampling members 130 and 130′ are situated in chambers 127 and 129, respectively.

FIG. 1E is a top view of a double-sided attachment member 125 that can be used to attach sampling member 130 to a selected surface in the environment at which microbial samples are to be gathered. FIG. 1F is a cross sectional view of attachment member 125 of FIG. 1E taken along section line 1F-1F. Attachment member 125 may be a double-sided adhesive tape such as shown in FIG. 1F or may employ hook-and-loop fasteners to adhere the attachment member 125 to a desired surface for sample collection purposes. As seen in FIG. 1F, double-sided attachment member 125 includes a flexible substrate 150 with adhesive layers 155 and 157 on the respective opposed surfaces thereof. Removable backing layers (i.e. release liners) 158 and 159 are situated on adhesive layers 155 and 157, respectively, to protect adhesive layers 155 and 157 prior to use.

FIG. 1G is a top view of a representative sampling member 130 that includes opposed surfaces of which upper surface 130A is visible in FIG. 1F. Sampling member 130 includes a negative control region 160 on upper surface 130A. Negative control region 160 is not exposed to the microbial sample being collected. Negative control region 160 may include a protective peel-off liner that a laboratory may peel off for inspection and testing if so desired. The peel-off liner of negative control region 160 is fabricated from a material impervious to microbes such as polyvinyl chloride (PVC) and other polymers. The protective peel-off liner (not shown) atop negative control region 160 provides a convenient place to hold sampling member 130 when placing sampling member 130 on a test surface. In summary, the portion of sampling member 130 directly below negative control region 160 is not exposed to the environment in which the microbial sample is collected by sampling member 130.

FIG. 1H is a cross-sectional view showing double-sided attachment member 125′ with backing layer 158 removed such that adhesive layer 155 adheres to bottom surface 130B of sampling member 130. After such removal of removable backing layer 158, the double-sided attachment member is designated double-sided attachment member 125′, as seen in FIG. 1H.

FIG. 1I is a cross-sectional view showing double-sided attachment member 125 with removable backing layer 159 removed such that adhesive layer 157 adheres to a surface 165 in the particular test environment selected for testing. In one embodiment, surface 165 may be inside of a face mask worn by a subject to collect microbial samples exhausted in the breath of the subject. Alternatively, surface 165 may be the exterior of the face mask for sampling external microbes from subject's environment or aerosols released by other person(s). In still other embodiments, surface 165 may be a surface in a room, an air duct of an HVAC system, or a surface in a hotel, a commercial enterprise, a government building, a vehicle, an airplane, a ship, a spacecraft or other surface for which microbial sampling is desired.

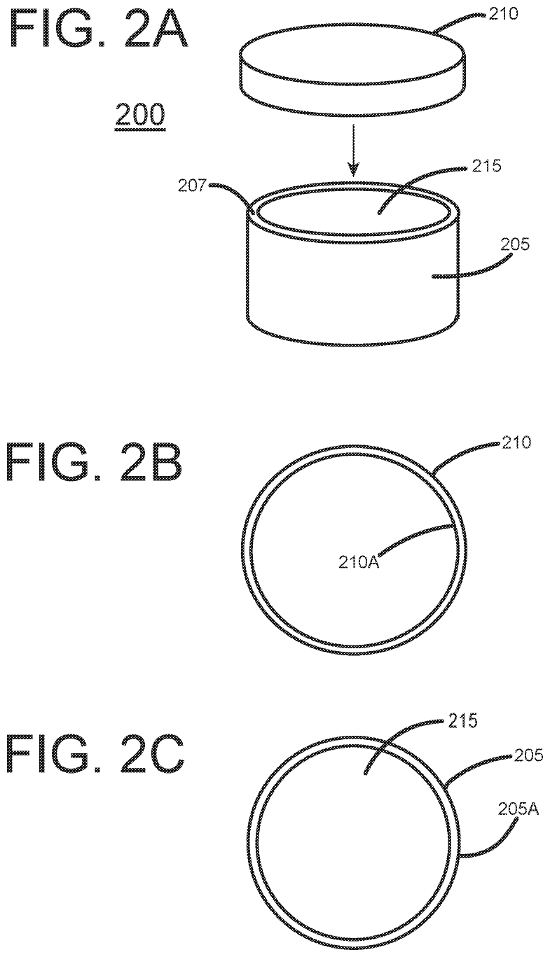

In another embodiment, a press-on (i.e. snap-on or pop-on) cap may be snapped onto a top of a main body to form an MSCTP container 200, as shown in FIG. 2A. More particularly, MSCTP container 200 includes a cylindrical main body 205 onto the top 207 of which a cap 210 may be snapped on and off. Main body 205 includes a chamber 215 into which at least one double-sided attachment member 125 and at least one sampling member 130 can be stored. In a manner similar to that of MSCTP container 100, MSCTP container 200 may include multiple partitioned chambers for storing respective double-sided attachment members 125 and sampling members 130. FIG. 2B shows a bottom view of snap-on cap 210 which includes an interior side wall 210A. FIG. 2C shows a top down view of main body 205 with its chamber 215. The interior wall 210A of snap-on cap 210 of FIG. 2B is dimensioned such that it snuggly fits over, and snaps on to, outer wall 205A of main body 205 of FIG. 2C.

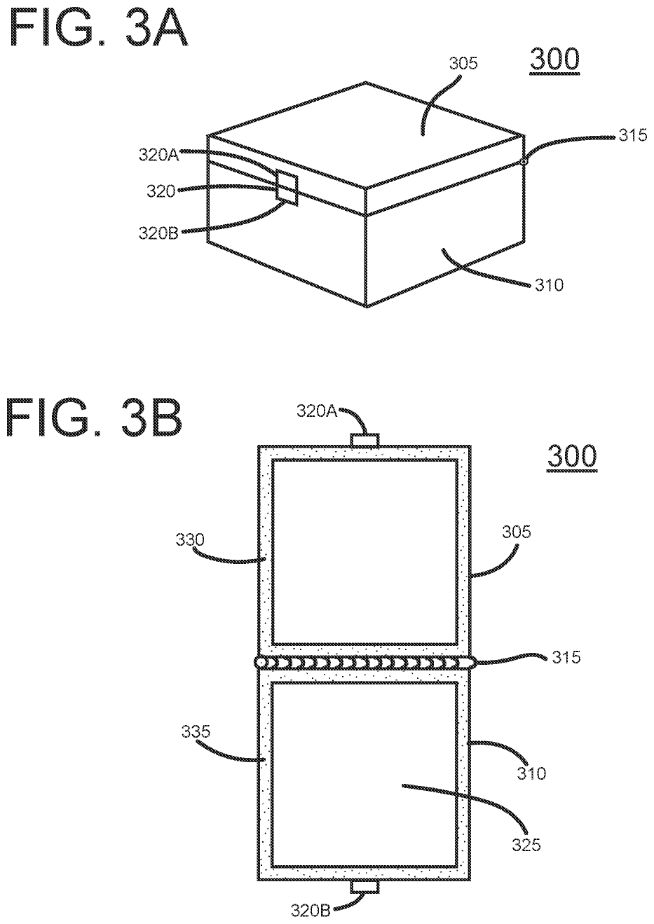

In another embodiment shown in FIG. 3A and FIG. 3B, an MSCTP container 300 exhibits a generally parallelepiped shape, such as square or rectangular. MSCTP container 300 includes a top portion 305, i.e. lid, that is hingably connected to bottom portion 310 via hinge 315. A latch 320 including upper latch 320A and lower latch 320B latch together to hold top portion 305 to bottom portion 310 together when MSCTP container is closed, as seen in FIG. 3A. In still another embodiment, the MSCTP container may be a substantially square or rectangular box that includes a retractable sliding door that opens and closes to allow placement of at least one double-sided attachment member and at least one sampling member in a chamber or chambers within the MSCTP container.

FIG. 3B shows MSCTP container 300 in the open state to reveal upper gasket 330 that attaches to the bottom perimeter of top portion 305, and to further reveal lower gasket 335 that attaches to the top perimeter of bottom portion 310. In this manner, when top portion 305 closes on bottom portion 310, gaskets 330 and 335 meet and compress together when latch 320 is latched. In this manner, MSCTP container 300 exhibits an air-tight and liquid tight seal when closed in one embodiment. While MSCTP container 300 is illustrated with a single chamber 325 therein, it should be understood that MSCTP container 300 may include multiple chambers in a manner similar to MSCTP containers 100 and 200, as described above.

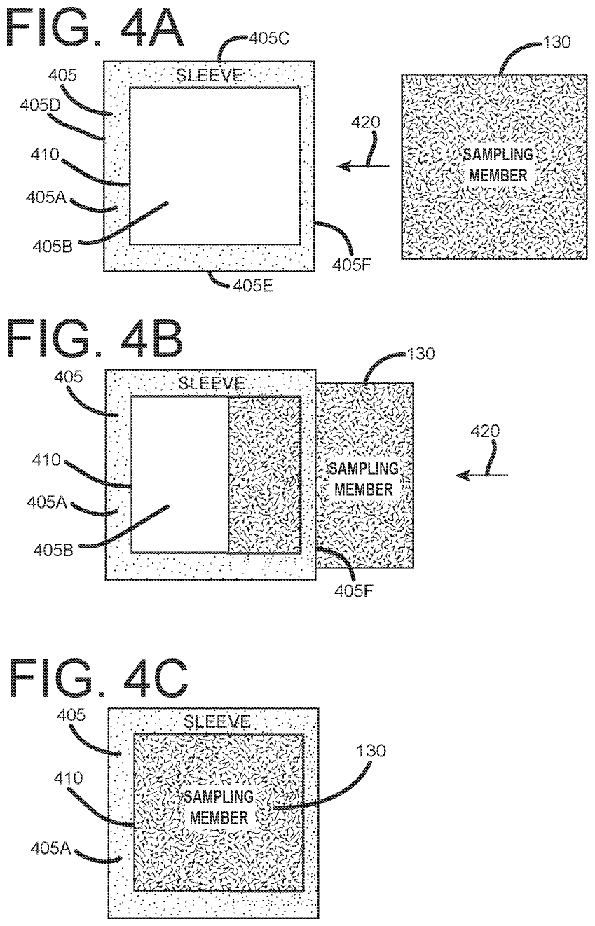

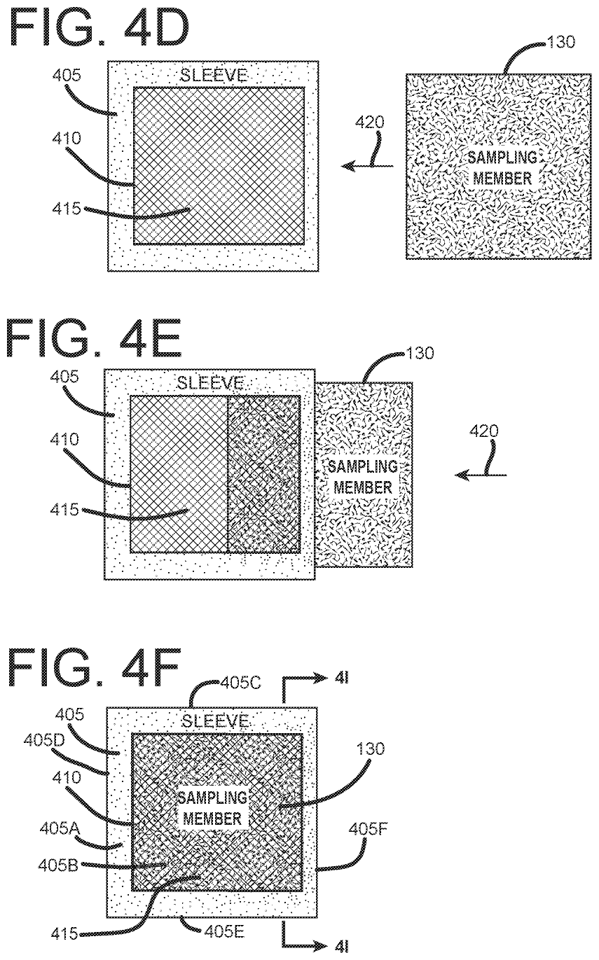

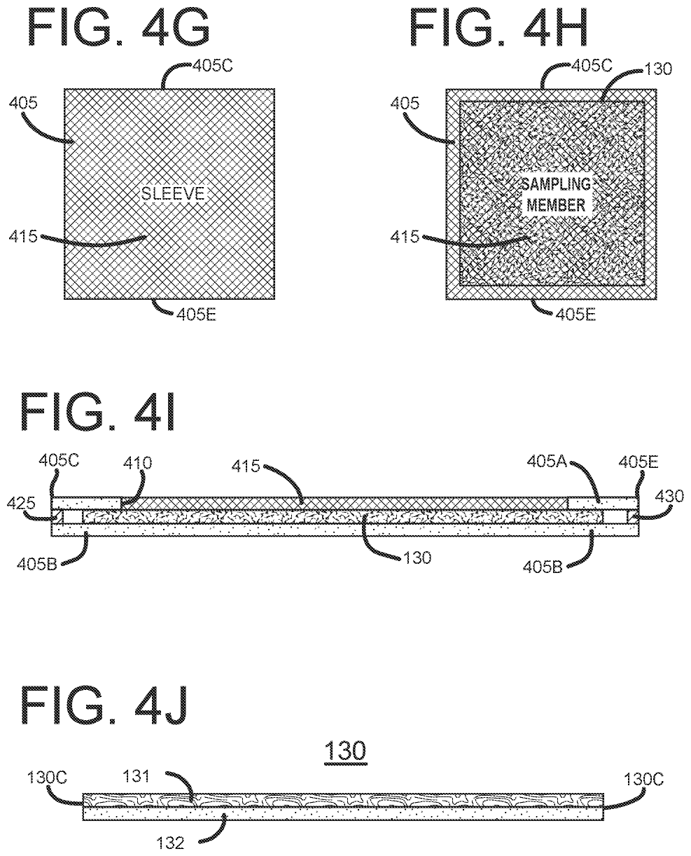

In the embodiment depicted in FIG. 4A-4C, rather than adhering sampling member 130 to a test surface in the test environment via double-sided attachment member 130, sampling member 130 may be placed in a protective sleeve 405 as shown. In this scenario, protective sleeve 405 with sampling member 130 therein is attached via double-sided attachment member 125 (not shown) to the test surface instead of directly attaching sampling member 130 to the test surface via double-sided attachment member 125 (not shown). In this embodiment, protective sleeve 405 includes a front panel 405A and an opposed back panel 405B that meet together on three (3) closed side edges, namely edges 405C, 405D and 405E. This configuration leaves side edge 405F open to receive sampling member 130 between front panel 405A and back panel 405C. Sleeve 405 may be fabricated from paper, polymer such as polypropylene, polycarbonate, and polyester such as rayon and nylon.

Protective sleeve 405 includes a window (i.e. an aperture) 410 that is open to expose sampling member 130 to the microbial samples to be collected. FIG. 4B shows sampling member 130 installed part of the way in sleeve 405. To fully install sampling member 130 in sleeve 405, sampling member 130 is moved in the direction of arrow 420 until sampling member 130 is completely within sleeve 405 as seen in FIG. 4C. Once sampling member 130 is fully installed in sleeve 405, the sample collector can adhere sleeve 405 to a selected test surface in the test environment and cumulative sample collection begins.

In the embodiment shown in FIGS. 4D-4F, window 410 is covered with a mesh 415 to provide sampling member 130 with more protection while still allowing sampling member 130 to be exposed to microbes which freely pass through mesh 415 to sampling member 130 thereunder. FIG. 4D shows sleeve 405 prior to the installation of sampling member 130 therein.

FIG. 4E shows sampling member 130 installed part of the way in sleeve 405. To fully install sampling member 130 in sleeve 405, sampling member 130 is moved in the direction of arrow 420 until sampling member 130 is completely within sleeve 405, as seen in FIG. 4F.

FIG. 4G shows an alternative embodiment wherein protective sleeve 415 is fabricated completely from mesh material to allow microbial samples to freely pass therethrough to a sampling member 130 inside sleeve 415 as seen in FIG. 4H.

FIG. 4I shows a cross section of sleeve 405 and sampling member 130 of FIG. 4F taken along section line 4I-41. As seen in FIG. 4F, sleeve 405 includes front panel 405A and back panel 405B. Back panel 405B is partially visible through mesh 415 that is on top of back panel 405B. Referring again to the cross section of FIG. 4I, mesh 415 is situated in window 410 with sampling member 130 positioned below mesh 415 and between front panel 405A and back panel 405B. Front panel 405A is adhered to back panel 405B via adhesive members 425 and 430 at side edges 405C and 405E, respectively, to form closed side edges. Front panel 405A is likewise adhered by an adhesive member (not shown) to back panel 405B at side edge 405D to close that side of sleeve 405. Thus, the side edges of front panel 405A and back panel 405B are coupled together via adhesive layers such as adhesive layers 425 and 430, with one edge 405F (FIG. 4) being left open to allow for the ingress and egress of sampling member 130. With sampling member 130 installed in sleeve 405 as seen in FIG. 4I, back panel 405B can be adhered by double-sided attachment member 125 (not shown) to the particular test surface at which microbe testing is desired.

In one embodiment, before sending MSCTP container 100 to a sample collector, sampling member 130 is pre-packaged inside sleeve 405 before placement of sampling member 130 in chamber 121 for shipment, as depicted in FIG. 4C. Double sided attachment member 125 can be stored in chamber 121 at the same time prior to shipment to the sample collector. In one embodiment, double-sided attachment member 125 is adhered to sampling member 130 prior to shipment of the MSCTP container and its contents to the sample collector. In this manner, when the sample collector receives the MSCTP container, the sampling member 130 is ready for attachment to a testing surface via double-sided attachment member 125. As noted, sampling member 130 may be shipped in MSCTP container 100 without sleeve 405. However, when a sleeve 405 is used, the back panel 405B of sleeve 405 may be removably adhered to a test surface where testing is desired via double-sided attachment member 125 that employs adhesive or hook and loop fasteners between back panel 405B and the selected test surface.

In another embodiment, sampling member 130 can be formed of a multi-layer set of filters that can size-wise exclude or separate particular microbes based on the size of the particular microbe. FIG. 4J shows such a sampling member 130 that includes a fine filter 131 and an ultra-fine filter 132. Fine filter 131 and ultra-fine filter 132 may alternatively be referenced as bacteria filter 131 and virus filter 132, respectively. Bacteria are physically larger than viruses. Fine filter 131 exhibits an appropriate micron rating to capture bacteria-size microbes and to allow virus-size microbes to pass therethrough. Ultra-fine filter 132 exhibits an appropriate micron rating to capture virus-size microbes or aerosol particles containing viruses.

In the embodiment of FIG. 4J, sampling member 130 includes a fine filter 131 situated atop an ultra-fine filter 132. Fine filter 131 is aligned with respect to, and removably attached to, ultra-fine filter 132 at the perimeter 130C of sampling member 130 as illustrated. This removable attachment is attained by situating a removable adhesive between fine filter 131 and ultra-fine filter 132 at perimeter 130C. In this manner, fine filter 131 can be easily removed from ultra-fine filter 132 for separate microbial testing by a lab technician or other tester. As noted, such adhesive may be placed around the entire perimeter of the multi-layer filter that forms sampling member 130. Alternatively, such adhesive may be applied to less than the 4 sides of sampling member 130 to provide the multi-layer filter. For example, adhesive may be applied to 3 sides, 2 sides or a single side of the perimeter to hold the multi-layer filter together. Applying the adhesive to 2 or more sides of the perimeter is typically preferred. Alternatively, the layers of the multi-layer filter, that in one embodiment form sampling member 130, may be stacked together without any adhesive and instead placing the filters in sleeve 405 to allow sleeve 405 to hold the filters together. It is noted that in another embodiment, instead of using mesh 415 in window 410 of protective sleeve 405, a coarse filter may be employed in place of mesh 415. Such a coarse filter would typically have a micron rating equal to or greater than approximately 10 microns and functionally would allow microbes and most human cells to pass through but capture dust particles.

Fine filter 131 excludes microbes exhibiting a size larger than a first particular selected microbe size. Fine filter 131 allows particles smaller than the particular selected microbe size to pass through to ultra-fine filter 132 and to be captured by ultra-fine filter 132 as a collected sample for later testing. Prior to testing fine filter 131 for a particular microbe, a lab technician separates fine filter 131 from ultra-fine filter 132 to expose ultra-fine filter 132. By way of example and not limitation, the multi-layered sampling member 130 can include a 0.45 micron fine filter 131 layered on top of a 0.25 micron ultra-fine filter 132. The 0.45 micron fine filter 131 will capture bacteria as samples while smaller viruses will pass through fine filter 131 and be captured on 0.25 micron ultra-fine filter 132 as samples. In this case, the lab technician separates fine filter 131 from ultra-fine filter 132 and then tests fine filter 131 and ultra-fine filter 132 separately.

In more detail, fine filter 131 is tested for the presence of a particular target bacteria and ultra-fine filter 132 is tested for the presence of a particular target virus. Processing multi-layered sampling member 130 in this manner enables the triage of different types of microbes improving the sensitivity of detection as well as triaging to different locations for microbe-specific testing. In one embodiment, filters 131 and 132 need not be adhered together at perimeter 130C if filters 131 and 132 are aligned as shown and stacked one atop the other and placed together inside protective sleeve 405 for sample collection. Alternatively, filters 131 and 132 need not be aligned while in protective sleeve 405. In yet another embodiment, a third filter layer (not shown) may be situated below ultra-fine filter 132 to capture microbial specimens as desired. More than 3 filters situated one atop the other may be used as well according to the desired application. In one embodiment, a fine filter may have a micron-rating between approximately 1 microns and approximately 10 microns, and an ultra-fine filter may have a micron-rating between approximately 0.2 and approximately 0.5 microns. It should be understood that these ranges are teachings intended as examples and should not be taking as limiting.

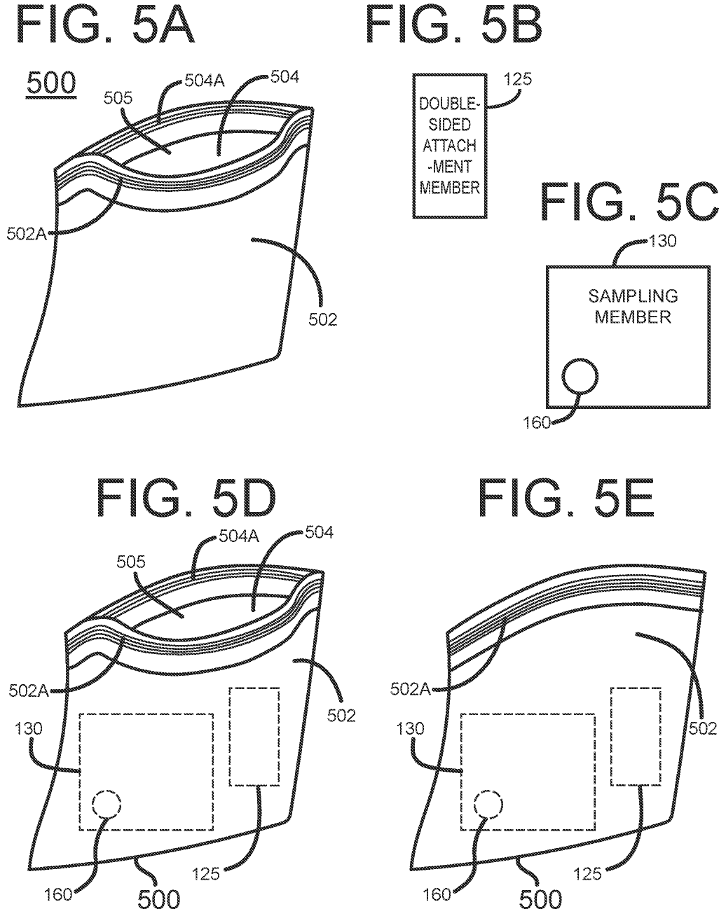

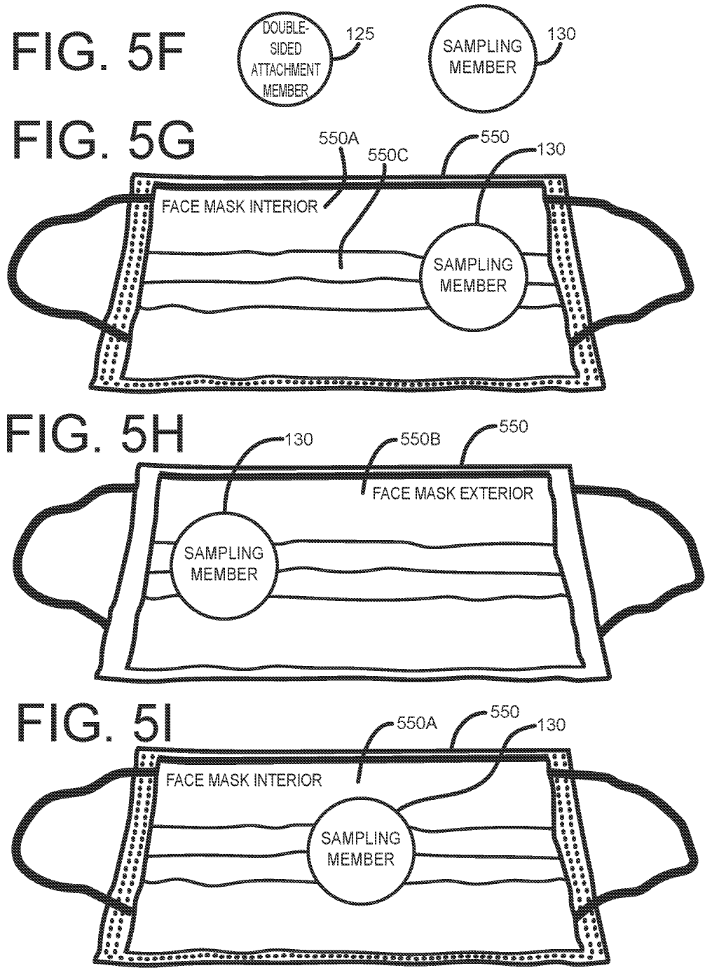

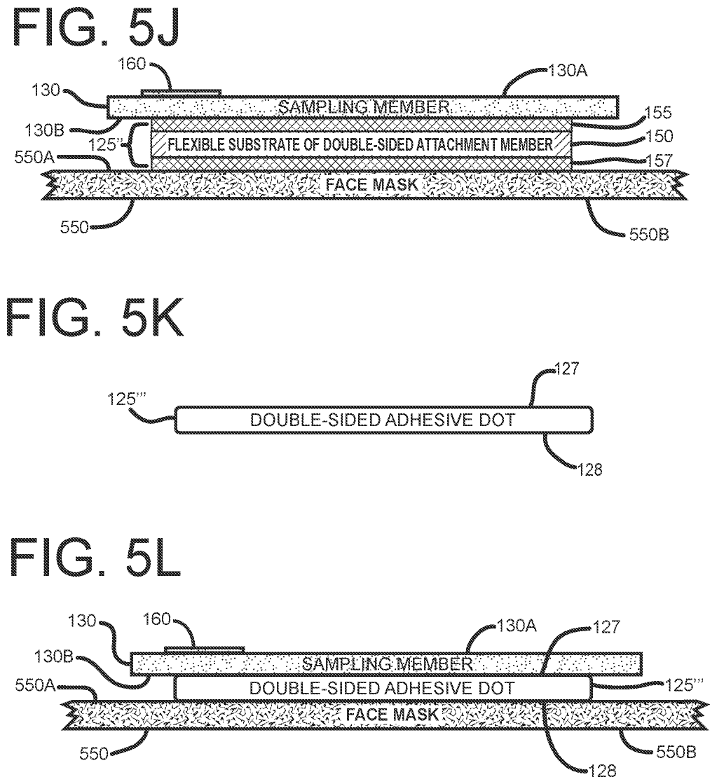

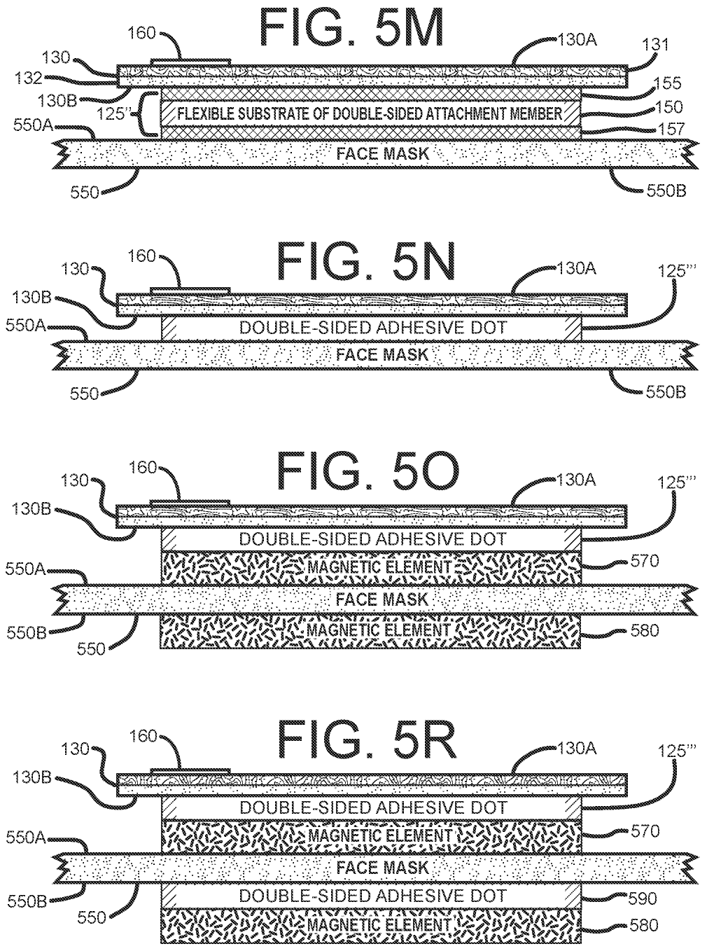

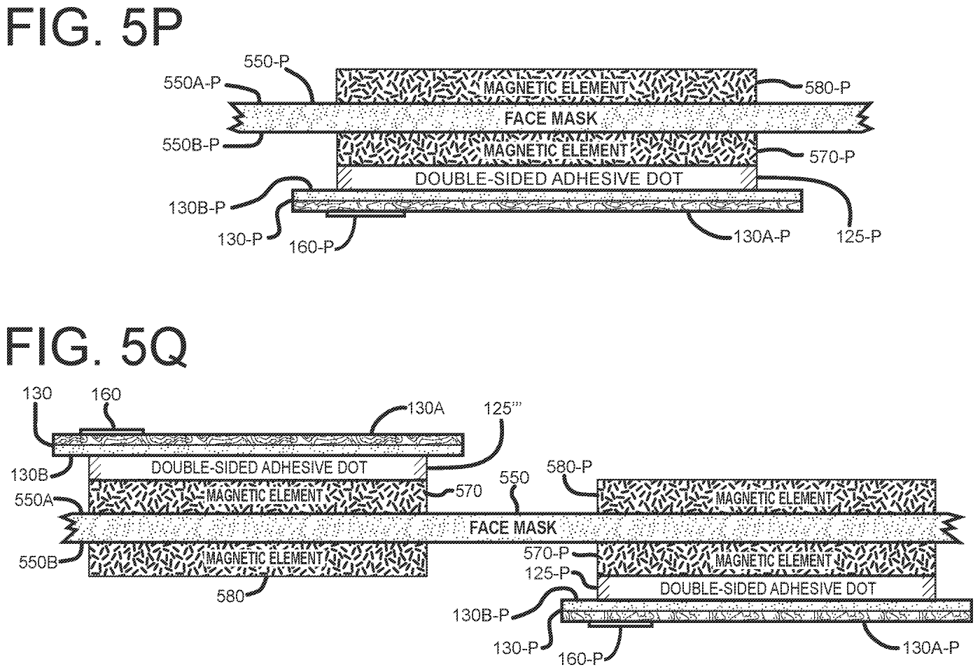

In FIG. 5A, a transparent, reclosable, resealable MSCTP container is depicted as container 500. MSCTP container 500 includes a front panel 502 and a back panel 504 that integrally meet together on 3 sides, as shown. The uppermost portion of front panel 502 includes a seal 502A that mates with a corresponding seal 504A on the uppermost portion of back panel 504 to close opening 505. FIG. 5B shows the double-sided attachment member 125 that is ready for placement in open MSCTP container 500. FIG. 5C shows the sampling member 130 that is ready for placement in open MSCTP container 500.

FIG. 5D shows MSCTP container 500 after a suppler or other entity places sampling member 130 and double-side attachment member 125 therein. The supplier or other entity then seals these contents inside MSCTP container 500 by mating seal 502A with seal 504A and pressing them together, thus closing and sealing MSCTP container 500 as seen in FIG. 5E. In this manner, an assembled kit is provided that is ready for use by a sample collector to cumulatively collect samples. The supplier or other entity sends the completed kit, namely MSCTP container 500 with double-sided attachment member 125 and sampling member 130 installed therein, to a sample collector who will engage in microbial sample collection at a location that the sample collector selects.

As seen in FIG. 5F, double-sided attachment member 125 and sampling member 130 are not limited to rectangular or square geometries. They may exhibit other geometries as well such as circular, oval and so forth. In the subsequent example, the kit assembled by the supplier includes a circular double-sided attachment member 125 and a circular sampling member 130 inside an MSCTP container 500 that is optionally transparent for easy viewing of the contents of the kit. In an alternative embodiment, MSCTP container 500 may be translucent or opaque. The MSCTP container 500 may also exhibit any color as desired. In another alternative embodiment, MSCTP container 500 can be sufficiently large to include not only double-sided attachment member 125 and sampling member 130, but to also accommodate a face mask 550 that is discussed below with reference to FIGS. 5G-5K.

The sample collector receives the kit from the supplier or other entity. The sample collector unseals MSCTP container 500 by opening it at the sealed end. The sample collector then removes double-sided attachment member 125 and sampling member 130 from MSCTP container 500. The sample collector adheres sampling member 130 to double-sided attachment member 125. Subsequently, the sample collector adheres the double-sided attachment member 125 to a surface in a selected test environment. For example, the sample collector may adhere sampling member 130 and double-sided attachment member 125 to the interior surface 550A of a facemask 550, as seen in FIG. 5G.

More specifically, the sample collector places sampling member 130 on facemask interior surface 550A at a location that will be adjacent the mouth and/or nose of the subject when the subject wears face mask 550. This configuration enables sampling member 130 to collect samples directly in the exhalation breath of the subject. In this manner, sampling member 130 will cumulatively collect microbial samples over a selected period of time, for example 1 day, 2 days or other desired period of time determined by the sample collector and/or the subject. In the embodiment of FIG. 5G, sampling member 130 is offset from the center 550C of mask interior surface 550A. It is noted that when the term “face mask” is used herein, the term is intended to include face coverings in general.

As seen in FIG. 5H, if desired, the sample collector can alternatively place sampling member 130 with double-sided attachment member 125 attached thereto on the exterior surface 550B of face mask 550. This enables sampling member 130 to cumulatively collect microbial samples for a selected period of time to determine exposure of the face mask 550 to microbes in the environment external to mask 550 as the subject wearing the mask moves from place to place in the subject's environment. The sample collector may also place respective sampling members 130 on both the interior 550A and exterior 550B of mask 550 to simultaneously monitor both locations for microbes.

As shown in FIG. 5I, the sample collector can alternatively place sampling member 130 via double-sided attachment member 125 to the center region of mask interior surface 550B. This region of face mask 550 is most directly in the exhalation breath path of the subject and sampling member 130 performs well to collect samples using this central location. However, the subject may be more comfortable with sampling member 130 being offset with respect to the center 550C of the face mask as seen in FIG. 5G.

FIG. 5J shows a side view of sampling member 130 attached via double-sided attachment member 125″ to the interior 550A of face mask 550. Broken lines are used in the illustration of face mask 550 to indicate that only a portion of face mask 550 is shown in FIG. 5J. In this manner, sampling member 130 is directly exposed to the subject's exhalation breath path to cumulatively collect microbial samples therefrom. As noted above, sampling member 130 and double-sided attachment member 125 may be placed at other locations in the environment for which cumulative microbial sample collection is desired, such as a room, an air duct of an HVAC system, or a surface in a hotel, a commercial enterprise, a government building, a vehicle, an airplane, a ship, a spacecraft or other surface for which microbial sampling is desired.

In another embodiment, double-sided attachment member 125 may be an adhesive dot 125′″, such as shown in FIG. 5K. Adhesive dot 125′″ is a two-sided adhesive member with removable properties. The opposed sides 127 and 128 of adhesive dot 125″ respectively readily adhere to surfaces and yet are easily removable therefrom. More particularly, as seen in FIG. 5L, side 127 of adhesive dot 125′″ adheres to lower surface 130B of sampling member 130. The sampling collector presses adhesive dot 125′″ to lower surface 130B to adhere these two elements together to form an assembly. The sampling collector takes this sampling member-adhesive dot assembly and applies this assembly to the interior surface 550A of face mask 550 at one of the locations described above. Sampling member 130 is now in position to commence collecting microbial samples in the breath path of a subject once the subject dons face mask 550.

In yet another embodiment, FIG. 5M shows a sampling member and double-sided attachment member assembly similar to that of FIG. 5J except that in the embodiment of FIG. 5N, the sampling member is a multi-layer filter 130 such as that described above with reference to FIG. 4J. In still another embodiment, FIG. 5N shows a sampling member and double-sided attachment member assembly similar to that of FIG. 5M, except that in the embodiment of FIG. 5N, the double-sided attachment member is a double-sided adhesive dot 125′″ that the sample collector adheres to face mask 550.

In still another embodiment, FIG. 5O shows sampling member 130 that adheres to a magnetic member 570 via a double-sided attachment member 125′″ therebetween. As one example, double-sided attachment member 125′″ may be a double-sided adhesive dot such as illustrated. The sample collector places another magnetic element 580 on the external surface 550B of face mask 550 at a location adjacent the location on the interior surface 550A of the facemask 550 where the sample collector desires to collect microbial samples. For example, the sample collector may want to collect samples adjacent the subject's nose and mouth in the subject's exhalation breath path inside facemask 550. To do so, the sample collector holds magnetic element 580 in position on the facemask external surface 550B adjacent the desired location on face mask interior surface 550A. The sample collector then moves magnetic element 570 with sampling member 130 attached closer and closer to the location on facemask internal surface 550A that is adjacent to the position of magnetic element 580 on facemask external surface 550B.

When magnetic element 570 comes sufficiently close to magnetic element 580, the magnetic force between magnetic elements 570 and 580 is sufficiently strong that magnetic element 570 is pulled toward magnetic element 580. In this manner, magnetic element 570 comes into contact with facemask internal surface 550A and is held thereto by the magnetic force between elements 570 and 580. This magnetic action effectively mounts magnetic element 570 with sampling member 130 attached thereto to the facemask internal surface 550A adjacent the location on facemask external surface 550B where the sample collector has been holding magnetic element 550 in position. The sample collector then discontinues holding magnetic elements 570 and 580 because magnetic element 570 with sampling member 130 is now magnetically held to magnetic element 580 via magnetic forces, thus effectively binding one to the other in a removable manner. As used herein, the term “adjacent” includes magnetic elements 570 and 580 being situated adjacent one another but on opposites sides of face mask 550. It is also noted that in FIG. 5O, magnetic elements 570 and 580 are substantially aligned with one another on the opposed sides of face mask 550, namely on face mask interior surface 550A and facemask exterior surface 550B, respectively.

It is noted that while both elements 570 and 580 are magnetic elements, i.e. they are both ferromagnetic, it is necessary for only one of elements 570 and 580 to be a magnet. In such an embodiment wherein only one of magnetic elements 570 and 580 is a magnet, it is necessary for the other magnetic element to be ferromagnetic so that one will attract the other. Examples of ferromagnetic materials include iron, steel, cobalt, nickel, alnico, ferrite and some compounds of rare earth metals. In another embodiment, both of magnetic elements 570 and 580 are magnets.

FIG. 5P shows an alternative embodiment similar to the embodiment of FIG. 5O except that the sample collector adheres magnetic element 580 to external facemask surface 550B via a magnetic element 570-P and a magnetic element 580-P as illustrated. Those components of FIG. 5P that are similar to components of FIG. 5A use the same component number except with the suffix “P” attached to the component number. FIG. 5Q shows an alternative embodiment that combines the teachings of FIG. 5O and FIG. 5P such that the attractive magnetic forces between magnetic elements 570 and 580 hold sampling member 130 in position adjacent the interior surface 550A of face mask 550, and at the same time the attractive magnetic forces between magnetic elements 570-P and 580-P hold sampling member 130-P in position adjacent the exterior surface 550A of the same mask 550. In this manner, as seen in FIG. 5Q, sampling members 130 and 130-P are magnetically held to the interior surface and exterior surface, respectively, of facemask 550. It is noted that the drawings herein are not drawn to scale. For example, the spacing between sampling member 130 and it associated elements on the left side of FIG. 5Q and sampling member 130-P on the right side of FIG. Q is typically more than shown. In actual practice, sampling member 130 and it associated elements are laterally spaced sufficiently far apart to not substantially interfere with the subject breathing through the facemask.

FIG. 5R shows an alternative embodiment similar to the embodiment of FIG. 5O except that the sample collector adheres magnetic element 580 to external facemask surface 550B via a double-adhesive attachment member 590 such as an adhesive dot. In this manner, once magnetic element 580 is secured to external surface 550B at the desired location, the sample collector need not continue to hold magnetic member 580 to external facemask surface 550B while moving magnetic element 570 into position on the internal facemask surface 550A adjacent magnetic element 580.

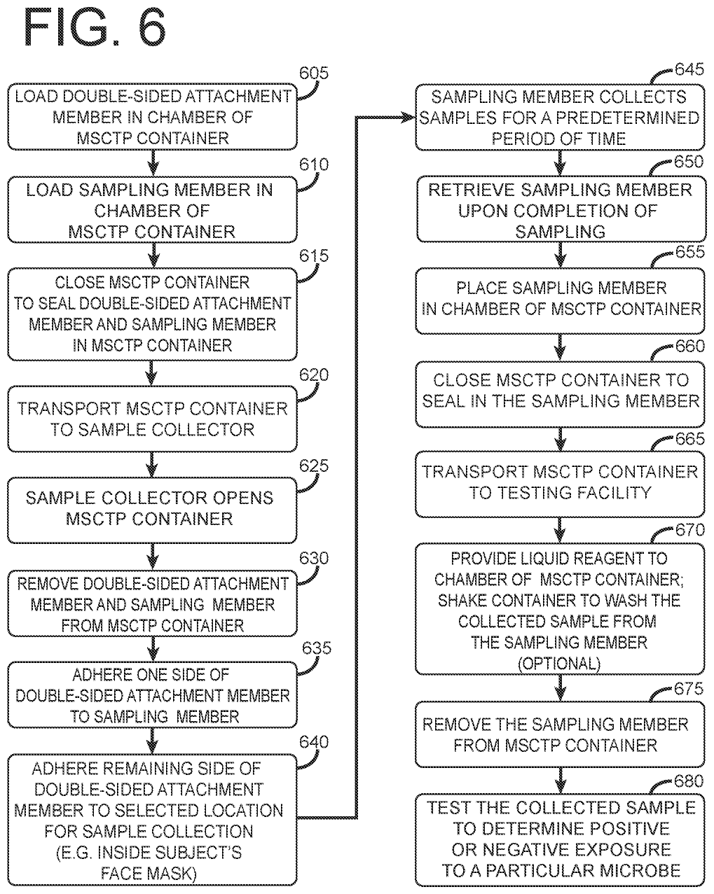

FIG. 6 is a flowchart that describes one embodiment of the disclosed microbial sample collection, transport and testing methodology. A worker, technician, or robotic machine at an MSCTP container kit assembly facility loads a double-sided attachment member 125 into chamber 121 of MSCTP container 100, as per block 605. The same entity loads sampling member 130 into chamber 121, as per block 610. Optionally, double-sided attachment member 125 is pre-attached to sampling member 130 at this point. MSCTP container 100 is closed to seal double-sided attachment member 125 and sampling member 130 in chamber 121, as per block 615.

MSCTP container 100 is transported to the location of a sample collector, as per block 620. The sample collector or other user opens MSCTP container 100, as per block 625. The sample collector or other user removes double-sided attachment member 125 and sampling member 130 from MSCTP container 100, as per block 630. The sample collector or other user attaches one side of double-sided attachment member 125 to sampling member 130 after removing the release liner from member 125, as per block 635. The sample collector or other user adheres the remaining side of double-sided attachment member 125 to a selected location for sample collection in a particular environment for which microbial testing is desired, as per block 640.

Sampling member 130 collects microbial samples at the selected location for a predetermined period of time, as per block 645. The sample collector or other user retrieves the sampling member 130 upon completion of collection, i.e. when the predetermined period of time has expired, as per block 650. The sample collector or other user removes the sampling member 130 from double-sided attachment member 125 and places sampling member 130 in chamber 121 within MSCTP container 100, as per block 655. Alternatively, sampling member 130 with double-sided attachment member 125 still attached thereto may together be placed in MSCTP container 100. The sample collector or other user closes MSCTP container 100 to seal sampling member 130 therein, as per block 660.

MSCTP container 100 with sampling member 130 therein is transported to a testing facility or location where equipment is available to test the collected sample, as per block 665. As an option, the sample collector may ship or drop off sampling member 130 in MSCTP container 100 at a local lab, pharmacy, sample collection kiosk or locker for pickup. When the MSCTP container 100 ultimately arrives at the test facility, a sample testing technician or lab technician can optionally commence a first step of processing the microbial sample on sampling member 130 by opening MSCTP container 100 and placing a liquid wash reagent into chamber 121. The technician closes MSCTP container 100 to reseal the sampling member 130 in chamber 121 of MSCTP container 100. The technician may agitate MSCTP container 100 to wash the microbial sample from sampling member 100, as per block 670 in this optional step. The technician removes sampling member 130 from MSCTP container 100, as per block 675. The technician tests the collected sample, whether it be in the optional liquid reagent wash, or still on sampling member 130, to determine the presence or absence of a particular microbial sample that is the target of the test, as per block 680. Those skilled in the art will appreciate that some or all of the above steps can be automated and need not necessarily be performed directly by a technician.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A microbial sample collection apparatus, comprising:
a first double-sided attachment member including first and second attachment surfaces;
a sampling member that is adhered to the first attachment surface of the first double-sided attachment member; and
a facemask including an interior surface configured to face a test subject, wherein the sampling member adheres to a selected location on the facemask interior surface via the second attachment surface of the first double-sided attachment therebetween.

2. The microbial sample collection apparatus of claim 1, wherein the first double-sided attachment member comprises an adhesive dot that adheres the sampling member to the selected location on the face mask interior surface.

3. The microbial sample collection apparatus of claim 1, wherein the sampling member includes a negative control region that protects a portion of the sampling member from being exposed to microbes.

4. The microbial sample collection apparatus of claim 1, wherein the sampling member includes a first filter layer situated atop a second filter layer, the first filter layer exhibiting a micron-rating sufficient to trap bacteria while allowing viruses to pass therethrough, the second filter layer exhibiting a micron-rating sufficient to trap viruses.

5. The microbial sample collection apparatus of claim 4, wherein the sampling member includes three or more filter layers.

6. The microbial sample collection apparatus of claim 1, further comprising a protective sleeve that houses the first double-sided adhesive member and sampling member therein.

7. The microbial sample collection apparatus of claim 1, further comprising a protective sleeve that houses the sampling member therein, the first double-sided attachment member being adhered to an external surface a protective sleeve.

8. The microbial sample collection apparatus of claim 6, wherein the protective sleeve includes a window that allows microbial samples to pass therethrough to the sampling member.

9. The microbial sample collection apparatus of claim 8, wherein the protective sleeve includes a mesh situated in the window that allows microbial samples to pass therethrough to the sampling member.

10. The microbial sample collection apparatus of claim 6, wherein the protective sleeve is a mesh material that protects the sampling member and the first double-sided attachment member therein and that allows microbial samples to pass through the mesh material to the sampling member.

11. The microbial sample collection apparatus of claim 1, wherein a sampling member is attached to an exterior surface of the facemask by a double-sided attachment member.

12. The microbial sample collection apparatus of claim 1, further comprising:
a first magnetic element adhered to the second attachment surface of the first double-sided attachment member and situated at the interior surface of the facemask,
a second magnetic element situated at an external surface of the facemask, wherein a magnetic force between the first and second magnetic elements and through the facemask is sufficiently strong to hold the sampling member with first magnetic element to the interior surface of the facemask at a predetermined location on the interior surface of the facemask adjacent the second magnetic element.

13. The microbial sample collection apparatus of claim 12, wherein the first double-sided attachment member is an adhesive dot.

14. The microbial sample collection apparatus of claim 1, further comprising:
a first magnetic element adhered to the second attachment surface of the first double-sided attachment member and situated at an exterior surface of the facemask,
a second magnetic element situated at the interior surface of the facemask, wherein a magnetic force between the first and second magnetic elements and through the facemask is sufficiently strong to hold the sampling member with first magnetic element to the exterior surface of the facemask at a predetermined location on the exterior surface of the facemask adjacent the first magnetic element.

15. The microbial sample collection apparatus of claim 1, further comprising:
a first magnetic element adhered to the second attachment surface of the first double-sided attachment member and situated at the interior surface of the facemask,
a second magnetic element situated at an exterior surface of the facemask, wherein a magnetic force between the first and second magnetic elements and through the facemask is sufficiently strong to hold the sampling member with first magnetic element to the interior surface of the facemask at a predetermined location on the interior surface of the facemask adjacent the second magnetic element.
a second double-sided attachment member including third and fourth attachment surfaces;
a second sampling member that is adhered to the third attachment surface of the second double-sided attachment member
a third magnetic element adhered to the fourth attachment surface of the second double-sided attachment member and situated at the exterior surface of the facemask,
a fourth magnetic element situated at the interior surface of the facemask, wherein a magnetic force between the third and fourth magnetic elements and through the facemask is sufficiently strong to hold the second sampling member with fourth magnetic element to the exterior surface of the facemask at a predetermined location on the exterior surface of the facemask adjacent the fourth magnetic element.

16. The microbial sample collection apparatus of claim 1, further comprising:
a first magnetic element adhered to the second attachment surface of first double-sided attachment member,
a second magnetic element adhered to an external surface of the facemask via a second double-sided attachment member therebetween, wherein the magnetic force between the first magnetic element and the second magnetic element is sufficiently strong to hold the sampling member to the interior surface of the facemask adjacent to the second magnetic element on the exterior surface on the facemask.

17. The microbial sample collection apparatus of claim 16, wherein the first double-sided attachment member is an adhesive dot and the second double-sided attachment member is an adhesive dot.

18. The microbial sample collection apparatus of claim 1 wherein a first sampling member is magnetically adhered to an interior surface of the facemask and a second sampling is magnetically adhered to an exterior surface of the facemask.

19. A microbial sample collection, transport and processing (MSCTP) container assembly, comprising:
an enclosure including an access region for adding and removing contents from the enclosure, the enclosure including at least one chamber therein for storing the contents;
a double-sided attachment member situated in the at least one chamber of the MSCTP container assembly as contents thereof; and
a sampling member situated in the at least one chamber of the MSCTP container assembly as contents thereof, the sampling member being configured to cumulatively collect microbial samples when installed on a selected surface via the double-sided attachment member.

20. The MSCTP container assembly of claim 19, wherein the access region is openable and closable.

21. The MSCTP container assembly of claim 19, wherein the access region is sealable and liquid-tight such that collected samples may be washed from the sampling member by a liquid reagent supplied to the chamber of the MSCTP container.

22. The MSCTP container assembly of claim 19, wherein the access region is sealable and air-tight.

23. The MSCTP container assembly of claim 19, wherein the sampling member is adhered to the double-sided attachment member.

24. The MSCTP container assembly of claim 19, wherein the sampling member is adhered, via the double-sided attachment member, to an interior surface of a face mask to be worn by a subject to collect microbial samples from a subject's breath.

25. The MSCTP container assembly of claim 19, wherein the sampling member is adhered, via the double-sided attachment member, to an exterior surface of a face mask to be worn by a subject to collect microbial samples in the subject's environment.

26. The MSCTP container assembly of claim 16, wherein the at least one chamber of the MSCTP container assembly includes a plurality of chambers, each chamber being configured to receive a different member associated with collecting microbial samples from a subject.

27. The MSCTEP container assembly of claim 19, wherein the MSCTP container assembly exhibits a generally cylindrical shape.

28. The MSCTP container assembly of claim 19, wherein the MSCTP container assembly exhibits a generally parallelepiped shape.

29. The MSCTP container assembly of claim 19, wherein the MSCTP container assembly is a plastic bag in which the access region includes a first seal that mates with a second seal to sealably enable ingress and egress of the contents.

30. The MSCTP container assembly of claim 19, wherein the sampling member includes a negative control region that is not exposed to microbial samples collected by the sampling member.

31. The MSCTP container assembly of claim 19, further comprising a main body and a cap that sealably screws on to the main body, the main body including the at least one chamber therein.

32. The MSCTP container assembly of claim 19, further comprising a main body and a press-on cap that sealably presses on to the main body, the main body including the at least one chamber therein.

33. The MSCTP container assembly of claim 19, further comprising a main body including the at least one chamber and a lid that hingably opens and closes on the main body, the lid including a first latch portion that mates with a second latch portion on the main body to sealably close the MSCTP container assembly.

34. The MSCTP container assembly of claim 16, wherein the MSCTP container assembly includes an identification region that identifies the subject whose samples are cumulatively collected by the sampling member.

35. The MSCTP container assembly of claim 34, wherein the identification region includes at least one of a bar code, a QR code or handwritten identification.

36. The MSCTP container assembly of claim 19, further comprising a protective sleeve in which the sampling member is stored.

37. The MSCTP container assembly of claim 36, wherein the protective sleeve exhibits a substantially rectangular geometry that is closed on 3 sides and open on a fourth side to receive the sampling member therein.

38. The MSCTP container assembly of claim 36 wherein the double-sided attachment member adhesively adheres to the protective sleeve via an adhesive layer therebetween.

39. The MSCTP container assembly of 36 wherein the double-sided attachment member adheres to the protective sleeve via first hook and loop fasteners therebetween.

40. The MSCTP container assembly of claim 36 wherein the double-sided attachment member adheres the protective sleeve with sampling member therein to a surface at a location where microbial testing is desired.

41. The MSCTP container assembly of claim 36, wherein the protective sleeve includes a window such that the sampling member therein is exposed to microbes.

42. The MSCTP container assembly of claim 39, wherein the window of the protective sleeve is covered with a mesh material such that the sampling member therein is exposed to microbes.

43. The MSCTP container assembly of claim 36, wherein the protective sleeve includes a subject identification region.

44. A method of processing samples, comprising:
placing, prior to microbial sample collection, a double-sided attachment member and a sampling member in a microbial sample collection, transport and processing (MSCTP) container, the MSCTP container including at least one chamber to receive the double-sided attachment member and the sampling member, the MSCTP container including an access portion that opens and closes to provide access to the at least one chamber;
opening, in preparation for microbial sample collection, the access portion of the MSCTP container to remove the double-sided attachment member and the sampling member therefrom;
adhering a side of the double-sided attachment member to the sampling member;
adhering another side of the double-sided attachment member to a surface within a breathing area of a subject such that the sampling member collects samples from the subject over a predetermined period of time, thus providing the sampling member with collected samples thereon;
moving the sampling member with samples thereon to the at least one chamber in the open MSCTP container; and
closing the access portion of the open MSCTP container with the sampling member having microbial samples thereon inside the MSCTP container, thus providing a closed MSCPT container.

45. The method of claim 44, wherein the sampling member includes a negative control region that is not exposed to the microbial sample being collected.

46. The method of claim 44, wherein the chamber is air-tight when the access portion is closed.

47. The method of claim 44, wherein the chamber is liquid-tight when the access portion is closed.

48. The method of claim 44, further comprising adhering the sampling member, via the double-sided attachment member, to an interior surface of a face mask to be worn by the subject to collect samples from the subject's breath.

49. The method of claim 44, wherein the MSCTP container includes a plurality of chambers, each chamber being configured to receive a different member associated with collecting samples from the subject.

50. The method of claim 44, further comprising opening the MSCTP container at a test facility and adding a liquid reagent to the chamber with the sampling member therein to wash collected microbial samples from the sampling member.

51. The method of claim 44, wherein the MSCTP container includes a first container portion in which the chamber is situated and a second container portion that sealably screws on to the first container portion, the method further comprising unscrewing the second container portion from the first container portion to open the MSCTP container and screwing the second container portion onto the first container portion to close and seal the MSCTP container.

52. The method of claim 44, wherein the MSCTP container includes a first container portion in which the chamber is situated and a second container portion that sealably mounts on the first container portion, the second container portion being a press-on cap.

53. The method of claim 44, wherein the MSCTP container includes a first container portion in which the chamber is situated and a second container portion, wherein the second container portion sealably closes to the first container portion, the second container portion including a lid that hingably opens and closes to cover a chamber of the first container portion below, wherein the lid includes a first latch portion that mates with a second latch portion situated on the second container portion to sealably close the MSCTP container, the method including latching the second container portion to the first container portion via the latch.

54. The method of claim 44, wherein the MSCTP container includes a first container portion in which the chamber is situated and a second container portion, the second container portion including a door that opens and closes to seal the access portion of the MSCTP container.

55. The method of claim 44, wherein the MSCTP container includes identification of the subject whose samples are collected by the sampling member.

56. The method of claim 44, wherein the identification is at least one of a bar code, a QR code or a handwritten identification.

57. The method of claim 44, further comprising inserting the sampling member in a protective sleeve that exhibits a substantially rectangular geometry that is closed on 3 sides and open on a fourth side to receive the sampling member therein.

58. The method of claim 44, wherein the double-sided attachment member adhesively adheres the protective sleeve, with the sampling member therein, to a testing surface.
